(19) United States
(12) Patent Application Publication
(10) Pub. No.: US 2023/0137627 A1
(43) Pub. Date: May 4, 2023

(54) METHOD FOR PRODUCING POLYMERSOMES

(71) Applicant: Heidelberg University, Heidelberg (DE)

(72) Inventors: Gert Fricker, Dossenheim (DE); Stefan Tobias Martin, Heidelberg (DE); Tobias Köthe, Heidelberg (DE)

(21) Appl. No.: 17/905,618

(22) PCT Filed: Mar. 22, 2021
(86) PCT No.: PCT/EP2021/057274

§ 371 Date: Sep. 2, 2022

(30) Foreign Application Priority Data
Mar. 20, 2020 (EP) 20164613.0

(51) Int. Cl. A61K 9/127; A61K 45/06

(52) U.S. Cl. CPC A61K 9/1273; A61K 45/06; A61K 9/1277

(57) ABSTRACT

The present invention relates to a method for producing polymersomes comprising a finalisation step using a dual centrifuge (DC) or a dual asymmetric centrifuge (DAC), polymersomes obtainable by said method and their use as a medicament.

### TECHNICAL FIELD

The present invention relates to a method for producing polymersomes comprising a finalisation step using a dual centrifuge (DC) or a dual asymmetric centrifuge (DAC).

### BACKGROUND OF THE INVENTION

As drug delivery systems, liposomes that are vesicles comprised of amphiphilic lipids are well established due their long clinical applications and known and tested characteristics. However, liposomes have many shortcomings, making them unsuitable as a drug delivery system in some cases. They are relatively susceptible to oxidation or hydrolysis and have a relatively low solubility, stability and half-life.

Polymersomes which are self-assembled polymeric vesicles made of amphiphilic block copolymers are promising alternatives to overcome the aforementioned shortcomings. Compared to the well-established and widely investigated liposomes, polymersomes can have multiple fold thicker membranes because of the higher molecular weight of block copolymers compared to phospholipids, which are commonly used as lipid membranes building blocks. This leads to a higher mechanical stability as well as a better protection of encapsulated water-soluble agents against leakage (A. Blanazs, S. P. Armes, A. J. Ryan, Self-Assembled Block Copolymer Aggregates: From Micelles to Vesicles and their Biological Applications, Macromol. Rapid Commun. 30 (2009) 267-277; G.-Y. Liu, C.-J. Chen, J. Ji, Biocompatible and biodegradable polymersomes as delivery vehicles in biomedical applications, Soft Matter 8 (2012) 8811).

The synthetic nature of block copolymers offers the possibility for functionalization such as for active targeting upon synthesis (F. Meng, C. Hiemstra, G. H. M. Engbers, J. Feijen, Biodegradable Polymersomes, Macromolecules 36 (2003) 3004-3006; D. E. Discher, F. Ahmed, Polymersomes, Annual Review of Biomedical Engineering 8 (2006) 323-341). Polymersomes have been found to have an up to two-fold longer circulation time in the blood-stream than, for example, PEGylated liposomes because of their higher PEG surface density and the ability to use PEG-chains with molecular weights >2000 Da which would lead to micelle formation when covalently bound to lipids (P. J. Photos, L. Bacakova, B. Discher, F. S. Bates, D. E. Discher, Polymer vesicles in vivo: correlations with PEG molecular weight, Journal of Controlled Release 90 (2003) 323-334). The mentioned advantages over liposomes make polymersomes interesting for the delivery of drugs. For this purpose, biocompatibility is required and biodegradability is preferred.

Medical applications of such drug delivery systems in humans, e.g. intravenous injection, generally require vesicle sizes below 200 nm to achieve colloidal characteristics with a polydispersity index (PDI) below 0.2, i.e., a homogeneous size distribution, which have been reported as being advantageous for various medical applications (S. A. Kulkarni, S.-S. Feng, Effects of particle size and surface modification on cellular uptake and biodistribution of polymeric nanoparticles for drug delivery, Pharmaceutical Research 30 (2013) 2512-2522.; T. Sun, Y. S. Zhang, B. Pang, D. C. Hyun, M. Yang, Y. Xia, Malßgeschneiderte Nanopartikel 540 für den Wirkstofftransport in der Krebstherapie, Angew. Chem. 126 (2014) 12520-12568). This includes, for example, overcoming the blood-brain barrier or the targeted control of tumours. Vesicle formation in this size range has so far only been possible if the polymer was dissolved in organic solvent and diluted with high volumes of aqueous solution (“solvent-switch” method, “direct-hydration” method).

Alternatively, high volumes of aqueous solution can be added to polymer films which, under prolonged exposure (24 h) to strong shaking and ultrasound, lead to vesicles of inhomogeneous sizes, and in which the vesicles then have to be brought to the correct size, usually by successive extrusion through different pore sizes, in a time-consuming and costly process (“film rehydration” method).

These previous methods for the production of polymersomes have many shortcomings (X. Sui, P. Kujala, G.-J. Janssen, E. de Jong, I. S. Zuhorn, J. C. M. van Hest, Robust formation of biodegradable polymersomes by direct hydration, Polym. Chem. 6 (2015) 691-696; J. E. Bartenstein, J. Robertson, G. Battaglia, W. H. Briscoe, Stability of polymersomes prepared by size exclusion chromatography and extrusion, Colloids and Surfaces A: Physicochemical and Engineering Aspects (2016) 739-746; M. Mohammadi, M. Ramezani, K. Abnous, M. Alibolandi, Biocompatible polymersomes-based cancer theranostics: Towards multifunctional nanomedicine, International Journal of Pharmaceutics (2017) 287-303)). For one, they use organic solvents during vesicle formation, which can induce instability of the active ingredients and thus lead to ineffectiveness or toxicity. Further, they require subsequent process steps for size reduction and standardization, which can lead to a loss of active ingredients enclosed in vesicles as the vesicles can open again. Moreover, in these methods, high volumes of dispersion medium or low polymer quantity and thus a low polymer concentration in the resulting vesicle dispersions are required, making the application of vesicle dispersions impractical or requiring additional concentration steps. Additionally, they yield low-concentration particle suspensions with an often broad size distribution of the resulting vesicles and are partly based on the use of organic solvents.

In view of the aforementioned shortcomings, it is therefore an object of the present invention to provide a novel method to produce small and homogeneous polymersomes in a simple manner in a high concentration without needing further size exclusion or extrusion steps downstream of the production step. It is a further object of the present invention to provide polymersomes obtainable by the method that meet the requirements outlined above.

### SUMMARY OF THE INVENTION

The present inventors have dedicated themselves to solving the problem of the present invention and were successful to find novel and useful methods to produce polymersomes which overcome the disadvantages and shortcomings of known methods. The aforementioned objects are solved by the method as defined by claim 1 and polymersomes as defined by claim 16. Advantageous developments are the subject matter of the dependent claims.

According to the first aspect of the present invention, a method for producing polymersomes is provided, comprising of the steps of preparing a mixture comprising an aqueous solvent, a block copolymer and a dispersing aid, optionally hydrating the copolymer in the mixture, and processing the mixture in a dual centrifuge (DC), preferably a dual asymmetric centrifuge (DAC), to obtain polymersomes.

According to a preferred embodiment of the first aspect of the present invention, a step of homogenizing the mixture is carried out before the step of processing the mixture, preferably wherein the step of homogenizing the mixture is carried out in a dual asymmetric centrifuge (DAC).

According to another preferred embodiment of the first aspect of the present invention, the duration of homogenizing is at least 1 minute, preferably at least 3 minutes, more preferably at least 5 minutes.

According to one preferred embodiment of the previous embodiment of the first aspect of the present invention, the speed of the dual asymmetric centrifuge in the step of homogenizing mixture is 2000 to 5000 rpm, preferably 3000 to 4000 rpm, more preferably 3400 to 3600 rpm, particularly preferably about 3540 rpm.

According to another preferred embodiment of the first aspect of the present invention, a step of hydrating is carried out for at least 10 minutes, preferably for at least 20 minutes, more preferably for at least 30 minutes.

According to yet another preferred embodiment of the first aspect of the present invention, the speed of the dual asymmetric centrifuge in the processing step is 2000 to 5000 rpm, preferably 3000 to 4000 rpm, more preferably 3400 to 3600 rpm, particularly preferably about 3540 rpm.

According to a further preferred embodiment of the first aspect of the present invention, the step of processing is carried out for a time between 10 and 50 minutes, preferably between 20 and 40 minutes, more preferably for about 30 minutes.

According to a preferred embodiment of the first aspect of the present invention, the mixture comprises 0.5 to 40 wt % block copolymer, 4.5 to 60 wt % aqueous solution and 20 to 95 wt % dispersing aid, preferably 2 to 20 wt % block copolymer, 10 to 50 wt % aqueous solution and 40 to 80 wt % dispersing aid, more preferably the mixture comprises 3 to 7 wt % block copolymer, 23 to 44 wt % aqueous solution and 50 to 73 wt % dispersing aid,

particularly preferably the mixture comprises 3.64 wt % block copolymer, 23.64 wt % aqueous solution and 72.73 wt % dispersing aid, or alternatively of 6.67 wt % block copolymer, 43.33 wt % aqueous solution and 50 wt % dispersing aid.

According to another preferred embodiment of the first aspect of the present invention, the block copolymer employed in the step of preparing a mixture is in a dehydrated state, more preferably the block copolymer is in the form of a copolymer film or a fine powder.

According to a preferred embodiment of the first aspect of the present invention, wherein the dispersing aid are beads made of ceramic, glass, metal or a composite material thereof.

According to yet another preferred embodiment of the first aspect of the present invention, the dispersing aid has an average particle size of 0.1 to 2 mm, preferably 0.6 to 1.6 mm, more preferably 0.8 to 1.4 mm, particularly preferably 1.0 to 1.2 mm.

According to another preferred embodiment of the first aspect of the present invention, the mixture is free from organic solvents and/or the block copolymer employed in the mixture is free from organic solvents and/or the processed mixture is free from organic solvents and/or the polymersomes obtainable by the method are free from organic solvents.

According to a further preferred embodiment of the first aspect of the present invention, the method for producing polymersomes does not include further extrusion steps.

According to another preferred embodiment of the first aspect of the present invention, the copolymer is block-copolymer, preferably a diblock-copolymer, more preferably a copolymer comprising polyethylene glycol und polycaprolacton (PEG-b-PCL).

According to one preferred embodiment of the first aspect of the present invention, the mixture prepared in step I. additionally comprises a substance or a pharmaceutically active ingredient suitable to be enclosed in or bound to the polymersomes obtained in step III, preferably wherein the substance or pharmaceutically active ingredient is hydrophilic.

According to a second aspect of the present invention, polymersomes are provided obtainable by the method according to the first aspect of the present invention.

According to another preferred embodiment of the second aspect of the present invention, the produced polymersomes are suitable for administration to a mammalian subject, preferably to a human subject.

According to yet another preferred embodiment of the second aspect of the present invention, the polymersomes are suitable for administration by intravenous or oral administration, preferably by intravenous administration.

According to yet another preferred embodiment of the second aspect of the present invention, the copolymer is a copolymer comprising polyethylene glycol und polycaprolacton (PEG-b-PCL)

According to one preferred embodiment of the second aspect of the present invention, the Z-Average size of the polymersomes is at most 1000 nm, preferably at most 600 nm, more preferably at most 400 nm, particularly preferably below 200 nm.

According to another preferred embodiment of the second aspect of the present invention, the polydispersity index PDI of the polymersomes is at most 0.5, preferably at most 0.3, particularly preferably at most 0.2.

According to yet another preferred embodiment of the second aspect of the present invention, the polymersomes additionally comprise a pharmaceutically active substance or a pharmaceutically active ingredient enclosed in or bound to the polymersomes.

According to one preferred embodiment of the previous embodiment of the first aspect of the present invention, the polymersomes are for use as a medicament.

### DESCRIPTION OF THE FIGURES

FIG. 1 schematically shows the method for producing polymersomes and the composition of three examples of mixtures used for producing polymersomes.

FIG. 2 shows transmission electron cryomicroscopy (Cryo-TEM) images of polymersomes produced by the method of the present invention.

FIG. 3 shows the encapsulation efficiency (EE) of different substances encapsulated in polymersomes made of PEG-b-PCL (5-b-20 kDa) and PEG-b-PCL (2-b-7.5 kDa) respectively.

FIG. 4 shows the total load of different substances encapsulated in polymersomes made of PEG-b-PCL (5-b-20 kDa) and PEG-b-PCL (2-b-7.5 kDa) respectively.

FIG. 5 shows CLSM images of the association of hypericin-laden polymersomes with Caco2 cells; (A) excitation: 561 nm, emission: 580-615 nm, (B) bright-field image of the same section.

FIG. 6 shows CLSM images of the association of rhodamin-laden polymersomes with Caco2 cells; (A) excitation: 561 nm, emission: 580-615 nm, (B) bright-field image of the same section.

FIG. 7 shows CLSM images of the association of hypericin-laden polymersomes with Caco2 cells in cross-section and detailed views; excitation: 561 nm, emission: 580-615 nm; on the right and on the bottom, reconstructed cross sections at the location of the index lines are shown.

### DETAILED DESCRIPTION OF THE INVENTION

The present invention is based on the recognition that polymersomes may be produced in an easy and advantageous manner by using dual centrifugation and that polymersomes produced in this manner can be used to encapsulate active drugs for applications in nanomedicine.

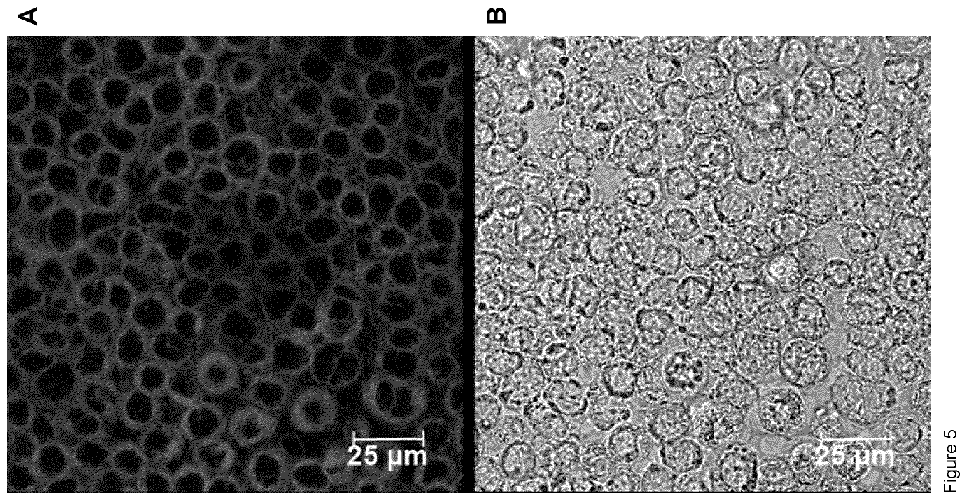

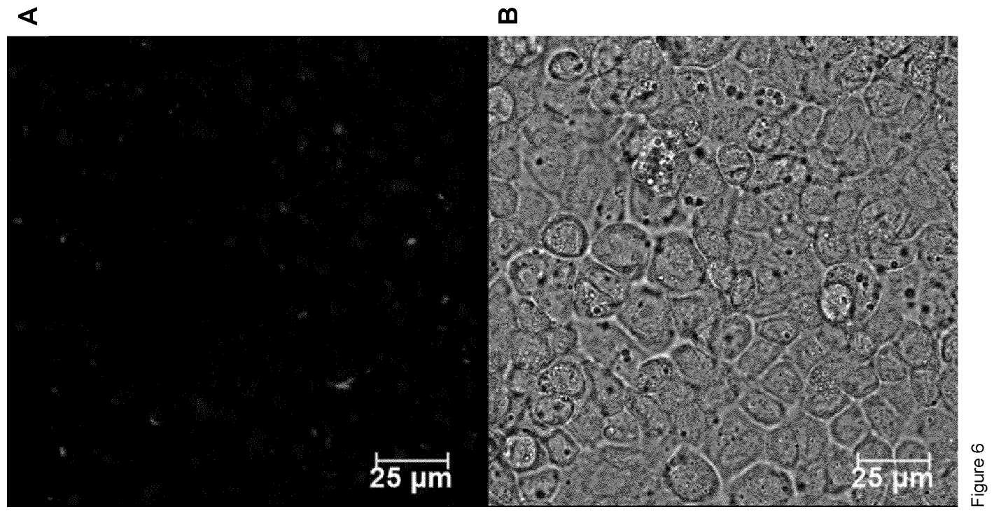

In contrast to liposomes, polymersomes obtained in this manner are able to specifically attach and adhere to colon cell surfaces without requiring additional modifications (cf. FIGS. 5 and 6). Such an adhesion can be advantageous per se e.g. for dosage forms targeting colon mucosa which are commonly used to release an active ingredient on site in case of inflammatory bowel disease.

Additionally, close contact with cell membranes also generally favors uptake of agents or particles into the interior of the cell. This is relevant whenever, for example, uptake in the intestine is to be achieved by entrapping an active ingredient in the vesicle, or when the blood-brain barrier should be transferred. In such a case, adhesion without additional modifications advantageously enables uptake in the next step.

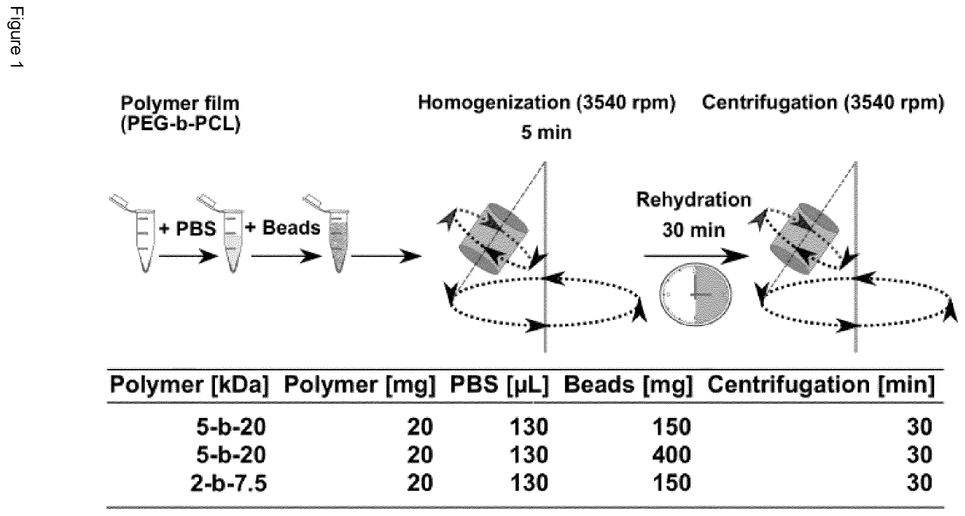

As shown in FIG. 1, the method comprises a step of preparing a mixture comprising an aqueous solvent, a copolymer and a dispersing aid, following optional steps of homogenizing the mixture and hydrating the copolymer in the mixture, and a subsequent step of processing the mixture prepared in a the previous steps in a dual centrifuge (DC), preferably in a dual asymmetric centrifuge (DAC), to obtain the polymersomes according to the invention.

In the step of preparing a mixture, the aqueous solution is preferably one or more of the group comprising water, a PBS buffered aqueous solution, a Tris buffered aqueous solution, a HEPES buffered aqueous solution, an aqueous solution of a drug to be encapsulated, or the like, more preferably a water-based salt solution of phosphate buffered saline (PBS) substantially comprising disodium hydrogen phosphate and sodium chloride, but may also be any other aqueous solvent.

Preferably, in the step of preparing a mixture, the copolymer is one of diblock copolymers polyethylene glycol-b-polycaprolacton (PEG-b-PCL), polyethylene glycol-b-polylactide (PEG-b-PLA), polyethylene glycol-b-poly(lactic-co-glycolic acid) (PEG-b-PLGA), polyethylene glycol-b-polyglycolid (PEG-b-PGA), poly(dimethylsiloxane)-b-poly(2-methyloxazoline) (PDMS-b-PMOXA), poly(3-caprolactone)-b-poly(2-methacryloyloxyethylphosphorylcholine) (PCL-b-PMPC), polylactid-b-poly(2-methacryloyloxyethylphosphorylcholine) (PLA-b-PMPC), polyethylene glycol-b-polybutadiene (PEG-b-PBD), polyethylene glycol-b-polyethylethylene (PEG-b-PEE), polyethylene glycol-b-polyphenylene sulfide (PEG-b-PPS), polyethylene glycol-b-polytrimethylene carbonate (PEG-b-PTMC) or the like, or triblock copolymers poly(lactic-co-glycolic acid)-b-polyethylene glycol-poly(lactic-co-glycolic acid) (PLGA-PEG-PLGA), poly(dimethylsiloxane)-b-poly(2-methyloxazoline)-b-poly(dimethylsiloxane) (PMOXA-b-PDMS-b-PMOXA), polyethylene glycol-b-polypropylene glycol-b-polyethylene glycol (PEG-PPO-PEG) or the like, more preferably the diblock copolymer polyethylene glycol-b-polycaprolacton (PEG-b-PCL).

The average polymer molecular weight fraction of the hydrophilic block portions of the copolymer is 14 to 45%, more preferably of about 20%. Within the context of the present invention, the average polymer molecular weight fraction of a block portion of the copolymer is the weight percentage relative to the total average polymer molecular weight of the copolymer.

Preferably, the copolymer is in form of a dry powder or a film that may be formed, for example, by dissolving the PEG-b-PCL in methylene chloride and evaporating said solution until the film is formed. Within the context of the present invention, the average polymer molecular weight fraction of a block portion of the copolymer is the weight percentage relative to the total average polymer molecular weight of the copolymer.

Furthermore, in the step of preparing a mixture, the dispersing aid may be spherical beads made of glass, metal or a composite material of different materials selected from the above, and volume average particle size diameters (d50) of the beads from 0.1 to 2 mm are preferred. More preferably, the dispersing aid may be spherical ceramic beads with volume average particle size diameters (d50) of 1.0 to 1.2 mm.

Within the context of the present invention, volume average particle size diameter (d50) is preferably analyzed using laser diffraction with Malvern Mastersizer 3000 Particle Size Analyzer as described in ISO Standard 13320 (2009) equipped with a hydro LV sampler and demineralized water as dispersant (Refractive Index=1.33). Material settings: a refractive index of 1.35, an absorption index of 0.60 and a density of 1 g/cm³. Sample is measured 3 times using continuous ultrasonic (setting at 50%) having a measurement loop of 30 sec using red light (630 nm) and 30 sec using blue light (470 nm). Average result will be reported as volume average particle size d50. D50 is defined as the particle size for which 50 percent by volume of the particles has a size lower than the d50.

Other methods for determining particle sizes may be used herein that are commonly known in the art and form part of the common general knowledge as shown in, for example, Kirk-Othmer, Encyclopedia of Chemical Technology, 4^{th} edition, John Wiley & Sons, New York (US), 1997, vol. 22, pages 256 to 278.

For the preparation of the mixture, preferably, a composition of the mixture comprising between 0.5 and 40 wt % copolymer, 4.5 and 60 wt % aqueous solution and 20 and 95 wt % dispersing aid, more preferred 3.64 wt % of copolymer, e.g., PEG-b-PCL, 23.64 wt % of aqueous solution, e.g., PBS and 72.73 wt % of dispersing aid, e.g., ceramic beads or another preferred composition of the mixture comprising 6.67 wt % of copolymer 43.33 wt % aqueous solution and 50 wt % of dispersing aid may be used, wherein wt % stands for mass fraction, i.e., percentage of the mass of an individual additive of the mixture relative to the total mass of the mixture.

Following the step of preparing a mixture, a step of homogenizing the mixture may preferably be carried out, in which the mixture is homogenized. As necessary, this step is more preferably carried out at any stage in the method before the step of processing the mixture. For carrying out the homogenization step, the prepared mixture is preferably disposed in a dual centrifuge (DC), more preferably in a dual asymmetric centrifuge (DAC), or any similar device.

DCs or DACs are characterized in that a sample, which is conventionally rotated about an rotation axis of a rotor to which the sample is arranged eccentrically in the rotor additionally rotates about its own rotation axis, in contrast to conventional centrifuges in which a sample is only rotated eccentrically about the rotation axis of the rotor in which it is disposed on. Through the second rotation about its own rotation axis, the sample is forced inwards towards the rotation axis of the rotor and thereby thoroughly mixed. DC and DAC differ in that, in a DC, the sample has a similar rotational direction as the rotor in which the sample is disposed on, whereas, in a DAC, a sample has a rotational direction substantially opposite to that of the rotor.

In the optional and preferred step of homogenizing the mixture, the mixture, after being disposed may then preferably subsequently be homogenized by being rotated with a rotational speed in terms of revolutions per minute (rpm). More preferably, the homogenization time during which the mixture is homogenized is at least 1 minute and the rotational speed is between 2000 and 5000 rpm. Particularly preferably, the homogenization time during which the mixture is homogenized is at least 5 minutes and the rotational speed by which the mixture is rotated is about 3540 rpm.

In the optional step of hydrating the copolymer in the mixture, preferably, the mixture is left at room temperature for 10 min or more after homogenization so that the PEG-b-PCL is hydrated before the step of processing the mixture. More preferably, the time the copolymer is hydrated is at least 30 minutes or the step of hydrating the copolymer in the mixture is omitted, as long as the PEG-b-PCL (or any other copolymer) is properly hydrated.

Preferably, in the step of processing the mixture, similar to the step of homogenizing the mixture described above, the mixture is disposed preferably in a DC, more preferably in a DAC. Consequently, the mixture is processed for at least 10 min by being rotated with a rotational speed of 2000 to 5000. More preferably, the time the mixture is processed is at least 20 minutes, particularly preferably 30 minutes, and the rotational speed by which the mixture is rotated is 3000 to 4000 rpm, particularly preferably about 3540 rpm.

The steps of homogenization of the mixture and the step of processing the mixture may preferably be carried out in a continuous fashion without an interruption. Alternatively preferably, the steps of homogenization and processing of the mixture are carried out as separate steps, wherein rotation is interrupted between the steps.

While processing the mixture, the individual copolymers in the mixture, particularly preferably the diblock copolymer PEG-b-PCL, self-assimilate as layers (usually monolayers in the case of triblock copolymers and bilayers in the case of diblock copolymers), consequently closing up spherically, thus forming polymersomes.

In this process of assimilating the polymersomes, prior to the completion of polymersome formation, a substance or pharmaceutically active ingredient is preferably added to the mixture suitable to be enclosed in or bound to the polymersomes. More preferably, the substance or the pharmaceutically active ingredient may be added to the mixture at any stage of the method described above.

As mentioned above, organic solvents may induce instability of some substances and active ingredients, which leads to ineffectiveness or toxicity when used for medical treatment. Therefore, preferably, the mixture is free from organic solvents and/or the copolymer employed in the mixture is free from organic solvents and/or the processed mixture is free from organic solvents and/or the polymersomes obtainable by the method are free from organic solvents. Within the context of the present invention, free from organic solvent preferably means a concentration of organic solvent of less than 10⁻⁴ g/ml, more preferably 10⁻⁵ g/ml, even more preferably less than 10⁻⁶ g/ml.

Regarding the parameters of the production method described herein, it is within the skill of the person skilled in the art to process the data disclosed herein, and optionally any additional data collected, using the MODDE® Pro 12 software (Umetrics, Umea, Sweden) in such a way that various parameter combinations are obtained which lead to the generation of advantageous polymersomes according to the invention.

The MODDE® Pro 12 software is designed for statistical design of experiments (DOE) and generation of valid models of parameter combinations and is based on the performance of automated multiple linear regressions over the collected experimental data.

By means of the experiments performed and the results obtained, a model was generated using the common general knowledge which was deemed valid according to the software referenced above. Said model reflects the influence of each of the parameters tested on the target variable (data not shown). Such a model is able to produce additional combinations of starting parameters within the so-called “design space” (defined by the starting parameters and the target variables obtained from them), which lead to target variables within the specified limits.

Next, the polymersomes obtainable by the method described above are described.

As described above, a substance or pharmaceutically active ingredient is preferably enclosed in or bound to the polymersomes assimilated in the step of processing the mixture, wherein the substance or pharmaceutically active ingredient is preferably hydrophilic. Preferably, the substance or pharmaceutically active ingredient is a peptide, more preferably a peptide comprising the amino acid sequence of SEQ ID No. 1

Seq ID No. 1

In 3-Letter Code

Tyr Pro Tyr Asp Val Pro Asp Tyr Ala Tyr
Pro Tyr Asp Val Pro Asp Tyr Ala Asp Ala
Glu Phe Gly His Asp Ser Gly Phe Glu Val
Arg His Gln Lys

In 1-Letter Code

Y P Y D V P D Y A Y P Y D V P D Y
A D A E F G H D S G F E V R H Q K

Alternatively preferably, the substance or pharmaceutically active ingredient is selected from ceftriaxone and hypericin.

Moreover, polymersomes are preferably suitable for administration to a mammalian subject, more preferably to a human subject. That is, the polymersomes may be, for example, biocompatible, more preferably biocompatible and biodegradable. Within the context of the present invention, biocompatible means that the polymersomes are non-toxic, do not have unwanted immunogenic properties and do not induce unwanted cellular alteration or degradation.

Furthermore, the polymersomes of the present invention are also disclosed for use as a medicament.

Preferably, the polymersomes are suitable as a depot or retard medication or for administration by intravenous, oral, buccal, nasal, sublingual or dermal administration, more preferably by oral or intravenous administration, particularly preferably by intravenous administration.

Preferably, the polymersomes have a Z-Average size of at most 1000 nm, more preferably at most 600 nm, even more preferably at most 400 nm, and a polydispersity index (PDI) of at most 0.5, more preferably at most 0.3. Particularly preferably, in regard to administration of the polymersomes into extracellular or intracellular space of a subject, i.e., systemic administration, the polymersomes have a Z-Average size of at most 200 nm and a PDI of at most 0.2, which is a requirement to be to be able to cross cell membranes and thus to be particularly interesting as a drug delivery system.

The Z-Average is measured by using dynamic light scattering and is a parameter defined by ISO 22412 as the “harmonic intensity averaged particle diameter” i.e. the average hydrodynamic particle size, whereas the polydispersity index (PDI) is a dimensionless number also calculated by using dynamic light scattering that describes the degree of non-uniformity of a size distribution of particles with values smaller than 0.05 indicate a highly monodisperse particle size and values bigger than 0.7 indicate a very broad particle size (Danaei, M.; Dehghankhold, M.; Ataei, S.; Hasanzadeh Davarani, F.; Javanmard, R.; Dokhani, A.; Khorasani, S.; Mozafari, M. R. Impact of Particle Size and Polydispersity Index on the Clinical Applications of Lipidic Nanocarrier Systems. Pharmaceutics 2018, 10, 57).

### Examples

### A) Specific Examples of the Method of the Invention

Preparing a Mixture

As copolymer, PEG-b-PCL with an average polymer molecular weight of 5-b-20 kDa and a PDI of 1.57 was used in form of dry powder or a film. The film was formed by dissolving the PEG-b-PCL in methylene chloride at 100 mg/mL in a 2 mL reaction tube and evaporated under nitrogen at 50° C. The residual solvent, in particular any organic solvent, was removed under vacuum for at least 1 h. As aqueous solution, PBS and, as dispersing aid, ceramic beads (SiLi Beads Type ZY-E 1.0-1.2 mm, Sigmund-Lindner GmbH, Germany) were used.

For preparing a mixture comprising PEG-b-PCL (5-b-20 kDa), 20 mg of PEG-b-PCL, 130 μL of PBS and 400 mg of ceramic beads were added together.

For preparing another mixture comprising PEG-b-PCL (5-b-20 kDa), 20 mg of PEG-b-PCL, 130 μL of PBS and 150 mg of ceramic beads were added together.

For preparing a mixture comprising PEG-b-PCL (2-b-20 7.5 kDa), 20 mg of PEG-b-PCL, 130 μL of PBS and 150 mg of ceramic beads were added together.

Homogenizing the Mixture and Hydrating the Copolymer in the Mixture

The resulting mixtures were disposed in a DAC and subsequently homogenized for 5 min at a rotational speed of 3540 rpm. After that, the mixtures were left at room temperature for 30 min to hydrate. This approach ensures that the PEG-b-PCL is properly hydrated.

Processing the Mixture in a DAC

After being homogenized and left for hydrating, the mixtures were disposed in the DAC and processed for 30 minutes at a rotational speed of 3540 rpm.

In the mixture comprising, 20 mg of PEG-b-PCL (5-b-20 kDa), 130 μL of PBS and 400 mg of ceramic beads polymersomes were yielded having a Z-Average size of 183±4 nm and a PDI of 0.140±0.003.

In the mixture comprising, 20 mg of PEG-b-PCL (5-b-20 kDa), 130 μL of PBS and 150 mg of ceramic beads polymersomes were yielded having a Z-Average size of 147±4 nm and a PDI of 0.083±0.007.

In the mixture comprising, 20 mg of PEG-b-PCL (2-b-7.5 kDa), 130 μL of PBS and 150 mg of ceramic beads polymersomes were yielded having a Z-Average size of 190±5 nm and a PDI of 0.27±0.01.

Polymersome Encapsulation

For the encapsulating step, polymersomes were prepared using the different mixtures of the method described above.

For one, Hypericin (HYP) and Ceftriaxone (CEF) as pharmaceutically active ingredient were encapsulated. To do so, 2 mg of Hypericin was dissolved in methylene chloride and added to the film prior to evaporation. Ceftriaxone encapsulating was performed by using Ceftriaxone solution of 400 mg/mL in distilled water instead of PBS as aqueous solution in the step of preparing a mixture.

As another substance, a 34 amino acid (AA) peptide with an amino acid sequence YPYDVPDYAYPYDVPDYADAEFGHDSGFEVRHQK (SEQ ID No. 1) was encapsulated by adding 2 mg of said peptide to the film prior to PBS addition or dissolving it as a 1.8 mg/mL solution in PBS.

### B) Devices and Experimental Methods

Dual Asymmetric Centrifuge (DAC)

The DAC used in the examples is a Speedmixer™ DAC 150 FVZ (Hauschild GmbH & Co KG, Hamm, Germany) with a distance between the rotation axis of the rotor and the rotation axis of the sample of 4.5 cm, a ratio of the rotation of the rotor and the rotation of the sample of approximately 4:1 and a maximum relative centrifugal force or g-force at the rotation axis of the sample of about 600.

Dynamic Light Scattering (DLS)

Using DLS, the produced Polymersoms were assessed for size and PDI with a Zetasizer Nano ZS (Malvern Instruments Ltd., Worcestershire, United Kingdom) equipped with a 633 nm laser at 173° backscattering. For calculating the mean z-average particle size and PDI, several measurements were taken and were measured using DLS.

Imaging by Transmission Electron Cryomicroscopy (Cryo-TEM Imaging)

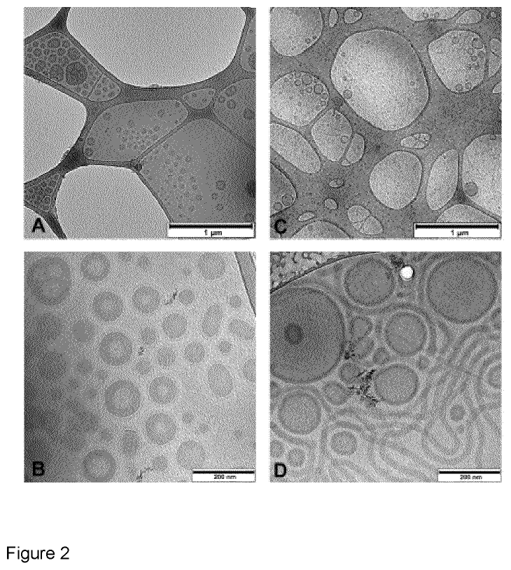

To adequately depict the morphology of nanoparticulate structures of the polymersomes, the polymersomes yielded from the different mixtures were examined using Cryo-TEM Imaging. To do this, a 4 μl aliquot of a sample of polymersomes was adsorbed onto holey carbon-coated grid (Lacey, Tedpella, USA), blotted three seconds with Whatman 1 filter paper and plunge-frozen into liquid ethane at −180° C. using a Vitrobot (FEI company, Hillsboro, USA). Frozen grids were transferred onto a CM FEG microscope (Philips, Amsterdam, Netherlands) using a Gatan 626 cryo-holder (GATAN, Pleasanton, USA). Electron micrographs were recorded at an accelerating voltage of 200 KV using low-dose system (20 to 30 e−/Å²) and keeping the sample at −175° C. Defocus values were −4 μm. Micrographs were recorded on 4K×4K TemCam-F CMOS based camera (TVIPS, Gauting, Germany). Nominal magnifications were 50,000× for high magnification images and 5,000× for low magnification images. To determine the dominant particle morphology, particles on low magnification images were counted and classified into monovesicular, solid and “other” depending on their morphology on the micrographs. Polymersomes wall thickness was evaluated by measuring pixel-thickness in GIMP 2.8 (https://www.gimp.org/) and converting to nm using the scale bar pixel-width. FIG. 2 shows images obtained by Cryo-TEM Imaging, wherein in A and B polymersomes and other structures based on PEG-b-PCL (5-b-20 kDa) and in C and D polymersomes and other structures based on PEG-b-PCL (5-b-20 kDa) are shown.

Separation by Size Exclusion Chromatography SEC

After being enclosed in polymersomes, substantially any free substance or ingredient was separated from polymersomes using SEC by applying 50 μL of each of the mixtures comprising polymersomes and the substances or ingredients to a gel filtration media in respective columns. The mixtures comprising HYP or CEF were applied to the gel filtration media Sephadex G-50 and the mixture comprising PEP was applied to the gel filtration media Sepharose CL-4B columns (inner diameter 15 mm, length 90 mm). Consequently, by hydrating and eluting the different columns with PBS, fractions of each column were collected, and fractionation was confirmed and substance or ingredient content was analyzed by using HPLC Analysis for PEP and CEF concentrations or Fluorescence Spectroscopy for HYP concentrations.

Determination of Encapsulation—HPLC Analysis

For determining concentrations of PEP and CEF in the fractions, an HPLC Agilent HP 0 system (Agilent Technologies, Palo Alto, Calif., USA) with UV detection on a reversed phase column was used. Curve fit was performed using 1/x weighted least squares linear regression (R²>0.99).

Fluorescence Spectroscopy

For determining concentration of HYP in the fractions, fluorescence spectroscopy (excitation: 540±25 nm, emission 590±20 nm) in a 96 well plate reader (Tecan Infinite Pro, Tecan Group Ltd., Männedorf, CH) was used. Curve fit was performed using unweighted least squares linear regression (R²>0.99).

Calculation of Encapsulation Efficiency EE and Load

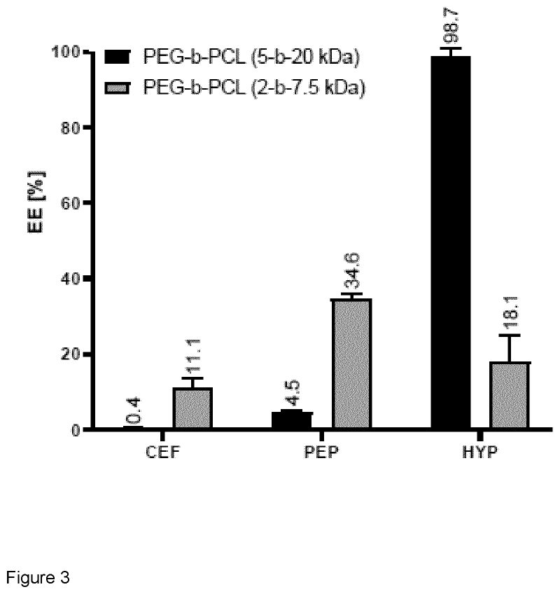

By means of the EE, FIG. 3 shows how much PEP, CEF and HYP were encapsulated by the polymersomes of the different mixtures. EE was calculated after correcting for all dilutions using the following equation:

EE [%]=100×(concentration of particle fraction)/(concentration of total sample)

The concentration of particle fraction is the concentration of the respective substance in the fraction obtained by SEC and the concentration of total sample the concentration of the substance initially set in the mixture.

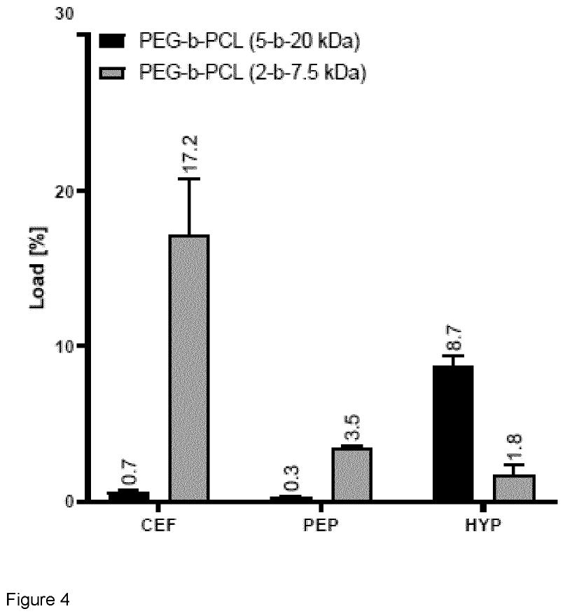

FIG. 4 shows the absolute load of the different mixture with the different substances, i.e., content of the respective substance relative to the mass of the copolymer, which was calculated using the following equation

Load [%]=100×((concentration of particle fraction)×(volume of particle fraction))/(mass of polymer)

The mass of polymer is the mass of the polymer in the fraction.

### C) Association of Polymersomes to Caco-2 Cells

Polymersome Preparation for Cell Experiments

Polymersomes were prepared using DAC (DAC 150 FVZ—modified to allow a maximum runtime of 30 min, Hauschild GmbH & Co KG, Hamm, Germany). 20 mg of PEG-b-PCL (2-b-7.5 kDa) was dissolved in methylene chloride at 100 mg/mL in a 2 mL Eppendorf tube and evaporated under nitrogen at 50° C. until a film was formed. 2 mg of hypericin was dissolved in isopropanol and added to the film prior to evaporation.

Residual solvent was removed under vacuum for at least 1 h. 130 μl Krebs-Ringer Buffer (KRB, for composition see Table 1) and 150 mg ceramic beads (SiLi Beads Type ZY-E 1.0-1.2 mm, Sigmund-Lindner GmbH, Germany) were added and samples were homogenized for 5 min by DAC at 3540 rpm. Samples were left at room temperature for 30 min to hydrate before again processing for 30 min with DAC at 3540 rpm. As hypericin itself is strongly fluorescent, these polymersomes do not have to be tagged using a different marker for being able to follow association by fluorescence microscopy.

Liposome Preparation for Cell Experiments

Liposomes consisting of 59.9 mole-% egg lecithin (Lipoid EPC-S), 40 mole-% cholesterol and 0.1 mole-% 18:1 Liss Rhod PE (NH4-Salt) were also prepared using DAC, using established methods. This results in a commonly used liposomal composition, representative of unmodified, non-fusogenic and non-cell penetrating liposomes, that has been widely used in literature as basic liposomes. The liposomal preparation is intensely fluorescent through the addition of 18:1 Liss Rhod PE, other properties are not changed significantly.

All lipids were dissolved as individual stocks in 9/1 chloroform/methanol as a solvent. To obtain the correct lipid composition, stocks were mixed in a 2 mL Eppendorf tube in the right proportions before evaporation under nitrogen at 50° C. until a film was formed. Residual solvents were again removed under vacuum. Ceramic beads (SiLi Beads ZY-E 1.0-1.2 mm, bead mass=6× lipid mass) and Krebs-Ringer Buffer (KRB, volume=1.5× lipid mass) was added to the lipid film before processing for 30 min in the DAC at 3540 rpm.

Afterwards, an additional volume of 2.5× lipid mass was added and the sample processed in the DAC for 5 min. This adding and processing step was done twice, resulting in a highly concentrated liposomal dispersion. This dispersion was then diluted with KRB to a final lipid concentration of 100 mM.

TABLE 1
Composition of KRB w/o Calcium as used here

| | | |
|---|---|---|
| Sodium chloride | 142 | mM |
| Potassium chloride | 3 | mM |
| Di-potassium hydrogen phosphate | 1.5 | mM |
| HEPES | 10 | mM |
| D-Glucose | 4 | mM |
| Magnesium Chloride | 1.2 | mM |

Measuring Cellular Association of Vesicles Using Confocal Laser-Scanning Microscopy (CLSM)

Liposome or polymersome dispersions were prepared as described and purified with size exclusion chromatography (PD SpinTrap G-25, according to the manufacturer's protocol 28-9180-04-5, spin protocol, equilibrated in KRB) to remove unencapsulated material or contaminants and diluted to the final concentration using KRB.

Caco-2 cells between passage 31 and 39 were used for all experiments. Cells were seeded in collagen-coated 8-well, glass bottom chambered coverslips (φ-Slides, ibidi GmbH, Gräfelfing, Germany) with a cell density of 1.3×10⁴ cells/cm². Cells were incubated for 4 days (37° C., 5% CO2 and 90% humidity). The medium was changed every other day. At the day of the experiment the medium was removed, and the cells were washed twice with KRB.

Afterwards KRB was applied and incubated for 15 minutes at 37° C. KRB was removed and 300 μL of each formulation (liposome concentration: 10 mM, polymersome concentration: 10 mg/ml) were applied and incubated for two hours at 37° C. in a drying oven. Following this, treatments were removed and cells were washed with 300 μL cold KRB. 300 μl cold acidic washing buffer (pH 3.0, 26 mM citric acid, 9.2 mM trisodiumcitrate, 90 mM sodium chloride, 30 mM potassium chloride) was added and incubated for 5 minutes at room temperature. After removal of the acidic washing buffer, 200 μL of KRB were added before measurement.

The liposomal membrane dye (18:1 Liss Rhod PE) and the hypericin contained in the polymersomes were measured with excitation 561 nm and emission at 580-615 nm. Pictures were taken using a Leica TCS SP5 confocal laser-scanning microscope (lens: PL APO 63.0×1.40 OIL, Pinhole[m]: 1 Airy unit, AOTF (488)—20%; AOTF (561)—1%; AOTF (633)—40%; Laser (Argon, visible) (Power) 20%). Photos in the same magnification were generally focused in the plane with the most visible fluorescence and taken at the same settings to ensure comparability.

Results of Polymersome/Liposome Cell Association Experiments

FIG. 5 and FIG. 6 show polymersomal and liposomal association to cells in comparison. It can be clearly seen that the fluorescent dye is surrounding each individual cell for the polymersome preparation. For unmodified liposomes, only minor random spots of fluorescence can be detected.

As the measurements were taken after washing steps that are designed to desorb loosely associated vesicles from cellular surfaces (acidic washing buffer) and to stop active uptake processes immediately (using ice cold washing solutions), the measured difference in association is immediately apparent and polymersomes are not only loosely, but strongly adsorbed onto all cellular surfaces.

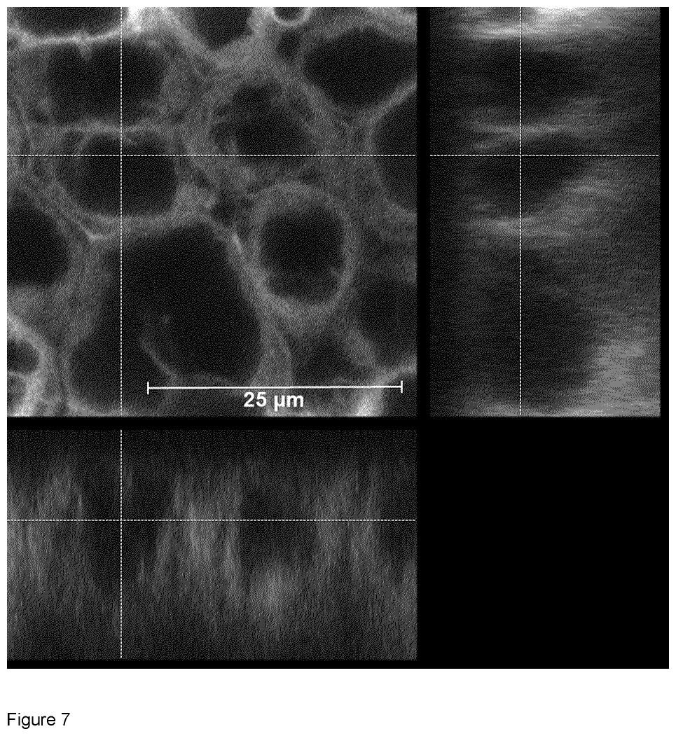

Using Confocal Laser Scanning Microscopy (CLSM), virtual cross sections of the sample were reconstructed using focus stacking of subsequent focal planes (FIG. 7). The resulting cross sections also clearly show association of polymersomes to cell membranes on all exposed areas of the cells, indicating efficient interaction with cellular surfaces.

1. A method for producing polymersomes, comprising the steps of
preparing a mixture comprising an aqueous solvent, a copolymer and a dispersing aid, optionally hydrating the copolymer in the mixture, and
processing the mixture in a dual centrifuge (DC), preferably a dual asymmetric centrifuge (DAC), to obtain polymersomes.

2. The method according to claim 1, wherein a step of homogenizing the mixture is carried out before the step of processing the mixture, preferably wherein the step of homogenizing the mixture is carried out in in a dual centrifuge (DC), more preferably a dual asymmetric centrifuge (DAC).

3. The method according to claim 2, wherein the duration of homogenizing is at least 1 minute, preferably at least 3 minutes, more preferably at least 5 minutes and/or wherein the speed of the dual asymmetric centrifuge in the step of homogenizing mixture is 2000 to 5000 rpm, preferably 3000 to 4000 rpm, more preferably 3400 to 3600 rpm, particularly preferably about 3540 rpm.

4. The method according to claim 1, wherein the copolymer employed in the step of preparing a mixture is in a dehydrated state, preferably wherein the copolymer is in the form of a copolymer film or a fine powder and/or wherein a step of hydrating is carried out for at least 10 minutes, preferably for at least 20 minutes, more preferably for at least 30 minutes.

5. The method according to claim 1, wherein the speed of the dual asymmetric centrifuge in the processing step is 2000 to 5000 rpm, preferably 3000 to 4000 rpm, more preferably 3400 to 3600 rpm, particularly preferably about 3540 rpm and/or wherein the step of processing is carried out for a time between 10 and 50 minutes, preferably between 20 and 40 minutes, more preferably for about 30 minutes.

6. The method according to claim 1, wherein the mixture comprises 0.5 to 40 wt % copolymer, 4.5 to 60 wt % aqueous solution and 20 to 95 wt % dispersing aid,
preferably wherein the mixture comprises 2 to 20 wt % copolymer, 10 to 50 wt % aqueous solution and 40 to 80 wt % dispersing aid, more preferably wherein the mixture comprises 3 to 7 wt % copolymer, 23 to 44 wt % aqueous solution and 50 to 73 wt % dispersing aid,
particularly preferably wherein the mixture consists of 3.64 wt % copolymer, 23.64 wt % aqueous solution and 72.72 wt % dispersing aid, or alternatively of 6.67 wt % copolymer, 43.33 wt % aqueous solution and 50 wt % dispersing aid.

7. The method according to claim 1, wherein the dispersing aid is highly spherical beads made of ceramic, glass, metal or a composite material thereof and/or wherein the dispersing aid has a diameter distribution with average diameters of 0.1 to 2 mm, preferably 0.6 to 1.6 mm, more preferably 0.8 to 1.4 mm, particularly preferably 1.0 to 1.2 mm.

8. The method according to claim 1, wherein the mixture is free from organic solvents and/or wherein the copolymer employed in the mixture is free from organic solvents and/or wherein the processed mixture is free from organic solvents and/or wherein the polymersomes obtainable by the method are free from organic solvents and/or wherein the method for producing polymersomes does not include further extrusion steps.

9. The method according to claim 1, wherein the copolymer is a block copolymer, preferably a diblock copolymer, more preferably a copolymer comprising polyethylene glycol and polycaprolacton (PEG b PCL).

10. The method according to claim 1, wherein the mixture prepared in step I. additionally comprises a substance or a pharmaceutically active ingredient suitable to be enclosed in or bound to the polymersomes obtained in step III., preferably wherein the substance or pharmaceutically active ingredient is hydrophilic.

11. Polymersomes obtainable by the method of claim 1, preferably wherein the polymersomes are suitable for administration to a mammalian subject, more preferably to a human subject and/or wherein the polymersomes are suitable for administration by intravenous or oral administration, preferably by intravenous administration.

12. Polymersomes according to claim 11, wherein the copolymer is a copolymer comprising polyethylene glycol und polycaprolacton (PEG b PCL).

13. Polymersomes according to claim 11, wherein the Z-Average size of the polymersomes is at most 1000 nm, preferably at most 600 nm, more preferably at most 400 nm, particularly preferably at most 200 nm and/or wherein the polydispersity index PDI of the polymersomes is at most 0.5, preferably at most 0.3, particularly preferably at most 0.2.

14. Polymersomes according to claim 11, wherein the polymersomes additionally comprise a pharmaceutically active substance or a pharmaceutically active ingredient enclosed in or bound to the polymersomes.

15. Polymersomes according to claim 14 for use as a medicament.

16. Polymersomes according to claim 14 for use in the treatment of diseases affecting the digestive tract, preferably inflammatory bowel diseases.


SEQUENCE LISTING (1 sequence)

SEQ ID NO: 1 - protein, 34 aa, Artificial Sequence
  Synthetic hydrophilic peptide
YPYDVPDYAYPYDVPDYADAEFGHDSGFEVRHQK